(19) United States
(12) Patent Application Publication
(10) Pub. No.: US 2015/0297873 A1
(43) Pub. Date: Oct. 22, 2015

(54) PERCUTANEOUS TUBE STABILIZATION DEVICE

(71) Applicants: CLEMSON UNIVERSITY RESEARCH FOUNDATION, Clemson, SC (US); GREENVILLE HOSPITAL SYSTEM, Greenville, SC (US)

(72) Inventors: Brennen Crenshaw Jenkins, Liberty, SC (US); Lauren Elizabeth Eskew, Mount Pleasant, SC (US); Breanne Therese Przestrzelski, Swannanona, NC (US); Carlyn Miller Atwood, Greenville, SC (US); Robert Gates, Greer, SC (US); John Chandler, Greenville, SC (US); John Desjardins, Clemson, SC (US)

(21) Appl. No.: 14/423,410

(22) PCT Filed: Aug. 22, 2013
(86) PCT No.: PCT/US2013/056204

§ 371 Date: Feb. 23, 2015

Related U.S. Application Data
(60) Provisional application No. 61/692,068, filed on Aug. 22, 2012.
(60) Provisional application No. 61/800,598, filed on Mar. 15, 2013.

(51) Int. Cl. A61M 27/00

(52) U.S. Cl. CPC A61M 27/00

(57) ABSTRACT

A stabilization device is described that can be utilized to properly align and secure a percutaneous tube such as a chest tube following insertion. The device includes a central passage that is ridged for a tight friction fit with a percutaneous tube, a base that includes features to improve contouring and flexibility of the base with the skin surface of a patient and eyelets for suturing to the skin that include insets for secure placement of the suture. The device can be used without need of adhesive and can hold a percutaneous tube at a comfortable exit angle without placing undue stress upon the sutures holding the device.

### BACKGROUND

Percutaneous tubes including drainage, vascular access and feeding tubes are commonly used in a wide variety of medical applications. For instance, chest tube insertions are the most commonly performed procedure in thoracic surgery. Unfortunately, due to the nature of percutaneous tube insertion, complications often arise following placement of the tube. For example, up to 30% of chest tube insertions involve complications that can include partial or complete dislodgment of the tube and for some disease processes, such as tension pneumothorax, accidental dislodgment of the chest tube can be catastrophic. In addition, it can be extremely costly, difficult and painful to have to undertake a repeat insertion procedure at a new site following dislodgement.

Percutaneous tube securement generally utilizes adhesives often in combination with suturing. Unfortunately, the use of adhesives has been associated with increased risk of infection. For instance, it is estimated that 12% of thoracostomy complications involve infection. When sutures are used, purse string suture or mattress suture closures are usually the suture of choice. In a purse string suture, a surgical suture is passed as a running stitch along the edge of the wound such that by drawing the two ends of the suture the wound is pulled around the tube. When utilizing a mattress stitch, the edges of the skin are pulled together tightly to form a smaller opening that abuts the tube wall. Unfortunately, these types of closures are complicated by the fact that they often fail to draw the tissue up tightly enough, leading to suture failure that leaves the tube unsecured. Sutures also must contain just the right amount of skin and subcutaneous tissue in order to be effective and secured properly, and both of these suture types are formed very near the cut edge of the skin, which can lead to loss of hold.

While external pressures due to contact and/or patient motion often lead to dislodgment of percutaneous tubes, the tubes can also dislodge merely under the force of gravity, particularly in those situations in which the body opening is larger than the tube and/or the tube is in contact with a large volume of ‘slick’ body fluids.

What are needed in the art are stabilization devices that can secure and properly align a percutaneous tube so as to better prevent dislodgement. A device that is relatively simple to manufacture and utilize, for instance a single piece device, would also be of great benefit.

### SUMMARY

Provided are devices for stabilizing a tube in the body of a subject. An example device includes a base portion having a proximal patient contacting surface and a top surface. The device further includes an elevated portion extending from the base portion and a passage defined by the base and elevated portions and having a passage wall. The passage extends from an opening at the distal edge of the elevated portion to an opening at the proximal edge of the base portion. The interior of the passage includes one or more textural features.

The one or more textural features are optionally configured to engage the tube such that advancement of the tube axially through the passage in the direction of the opening at the proximal edge of the base portion produces less resistance than retraction of the tube axially through the passage in the direction of the opening at the distal edge of the elevated portion. Optionally, the passage is sized to allow for slideable advancement of the tube axially through the passage in the direction of the opening at the bottom of the base portion while the tube is engaged by the textural features.

Optionally, the device is formed of a biocompatible material. For example, the device is optionally formed from a biocompatible silicone elastomer. Optionally, the silicone elastomer has a Shore A hardness of from about 30 to about 60, as measured by DIN 53505. Optionally, the silicone elastomer has a tensile strength of from about 6 to about 12 N/mm² as measured by DIN 53504 S 1. Optionally, the silicone elastomer has a tear strength of from about 20 N/mm to about 70 N/mm, as measured by ASTM D624 B.

The passage lumen optionally has a circular transverse cross-section. The one or more textural features optionally include one or more protrusions extending from the passage wall into the passage lumen, each protrusion including a point in maximal proximity to the central axis of the passage. The one or more protrusions are optionally one or more ridges. For example, the textural features can comprise a plurality of ridges. The ridges are optionally parallel. The ridges are optionally arranged perpendicular to the central axis of the lumen. The ridges optionally have a pitch relative to the axis of the lumen. Optionally, the ridges are continuous around the interior surface of the lumen. Optionally, the ridges are discontinuous around the interior surface of the lumen.

The relief of the protrusions, measured as the distance from the point in maximal proximity to the central axis to the plane of the interior surface of the passage, optionally ranges from about 0.05 mm to about 0.75 mm. Optionally, the relief of the protrusions, measured as the distance from the point in maximal proximity to the central axis to the plane of the interior surface of the passage, is from about 1% to about 10% of the largest cross-sectional dimension of the opening at the top of the elevated portion. Optionally, the relief of the protrusions, measured as the distance from the point in maximal proximity to the central axis to the plane of the interior surface of the passage, is from about 2.5% to about 8% of the largest cross-sectional dimension of the distal opening of the lumen.

Each protrusion optionally includes a base at the passage lumen wall. The base includes a proximal point and a distal point. The proximal point is the point at which the base is in maximum proximity to the opening at the bottom of the base portion. The distal point is the point at which the base is in maximum proximity to the opening at the top of the elevated portion. Optionally, the point in maximal proximity to the central axis is positioned closer to the opening at the bottom of the base portion than the proximal point.

The height of the protrusions, measured as the distance from the proximal point to the distal point is optionally from about 0.15 mm to about 1.5 mm. The length of the passage is optionally from about 3 mm to about 50 mm. The largest cross-sectional dimension of the opening at the top of the elevated portion is from 90% to 99% of the outer diameter of the tube. The largest cross-sectional dimension of the opening at the top of the elevated portion is optionally from 0% to 10% smaller than the largest cross-sectional dimension of the opening at the bottom of the base portion. Optionally, the central axis of the lumen is perpendicular to the plane of the base portion. Optionally, the central axis of the lumen is angled with respect to the plane of the base portion. For example, the angle of the central axis of the lumen with respect to the plane of the base can be varied from vertical to accommodate tube angulation with respect to the skin and chest exit. Optionally, the base portion further comprises a plurality of anchor points. For example, the anchor points optionally include eyelets or hooks. Optionally, the anchor points are positioned symmetrically around the base portion. The eyelets can be, for example, filed with a polymer membrane which can be pierced during attachment of the sutures.

The height of the elevated portion, as measured from the top surface of the base portion to the top of the elevated portion, is optionally from about 5 mm to about 50 mm. Optionally, the elevated portion further comprises a retaining element positioned in proximity to the top of the elevated portion.

The example device optionally further comprises an adhesive disposed on the patient contacting surface of the base portion. Optionally, the base portion further comprises a plurality of indentations. Optionally, a channel is disposed within the elevated portion in proximity to the top surface of the base portion. The height of the channel is optionally from about 0.1 mm to about 1.5 mm.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 is a perspective view of a stabilization device as described herein.

FIG. 2 is a top view of a device.

FIG. 3 is a partial side view of a device.

FIG. 4 is a cross-sectional view of a device.

FIG. 5 illustrates a portion of a device in a perspective view.

FIG. 6 illustrates a device in conjunction with a percutaneous tube.

FIGS. 7A-7D illustrate a procedure for utilization of a stabilization device.

FIG. 8 is an enlarged cross-sectional view of the ridge extending from the passage wall into the passage lumen.

FIG. 9 is a cross-sectional view of a device.

FIG. 10 is a cross-sectional view of a device.

FIG. 11 is a cross-sectional view of a device.

FIG. 12 is a cross-sectional view of a device.

FIG. 13 is a cross-sectional view of a device.

FIG. 14 is a cross-sectional view of a device.

### DETAILED DESCRIPTION

Reference will now be made in detail to various embodiments of the disclosed subject matter, one or more examples of which are set forth below. Each embodiment is provided by way of explanation of the subject matter, not a limitation of the subject matter. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment. Thus, it is intended that the present disclosure cover such modifications and variations as come within the scope of the appended claims and their equivalents.

The present disclosure is generally directed to a stabilization device for percutaneous tubes. More specifically, the disclosed device can be utilized to properly align and secure a percutaneous tube following implantation. In one specific embodiment, the stabilization device can be utilized to properly align and secure a thoracostomy tube within the pleural cavity. Tube thoracostomy is a procedure that is used to drain the pleural space of air, mucus, blood, or any other fluid. It should be understood, however, that while the disclosed stabilization device may prove exceedingly beneficial when utilized in conjunction with a thoracostomy tube, disclosed devices are in no way limited to utilization with chest tubes, and the device may be utilized to stabilize any percutaneous tube including, without limitation, surgical drainage tubes, gastronomy tubes, Y-shaped cardiac drainage systems, vascular access tubes, central lines, venous and arterial access ports, colostomy tubes, and so forth.

The stabilization device can increase the stability of the percutaneous tube and thereby decrease the likelihood of partial or complete dislodgement while also reducing the risk of infection. The stabilization device can be firmly secured in place by the use of sutures alone, without the need of adhesives in conjunction with the sutures. Moreover, the device can be secured with fewer sutures than have been utilized for suture securement in the past (e.g., a mattress suture or purse string suture technique). As such, use of the device can reduce complications associated with sutures such as risk of air leakage, skin necrosis, and poor cosmetic results. Adhesives can cause irritation as well as create a potential infection at the insertion site. Thus, elimination of the need for adhesives can increase the safety of the insertion process. The device can further comprise a silver impregnated alginate, which is optionally used to prevent infection.

The stabilization device offers additional benefits as well. Many currently utilized securement devices are anchored at a distance from the tube insertion site, which allows for undesired motion of the tube at the insertion site that can lead to dislodgement as well as other undesirable consequences such as pain and tissue damage. Disclosed stabilization devices are designed to fit over the percutaneous tube and be secured to the patient at the insertion site. This can decrease the distance between the tube insertion site and the anchoring site, thereby reducing potential movement of the tube both at the insertion site and subdermally.

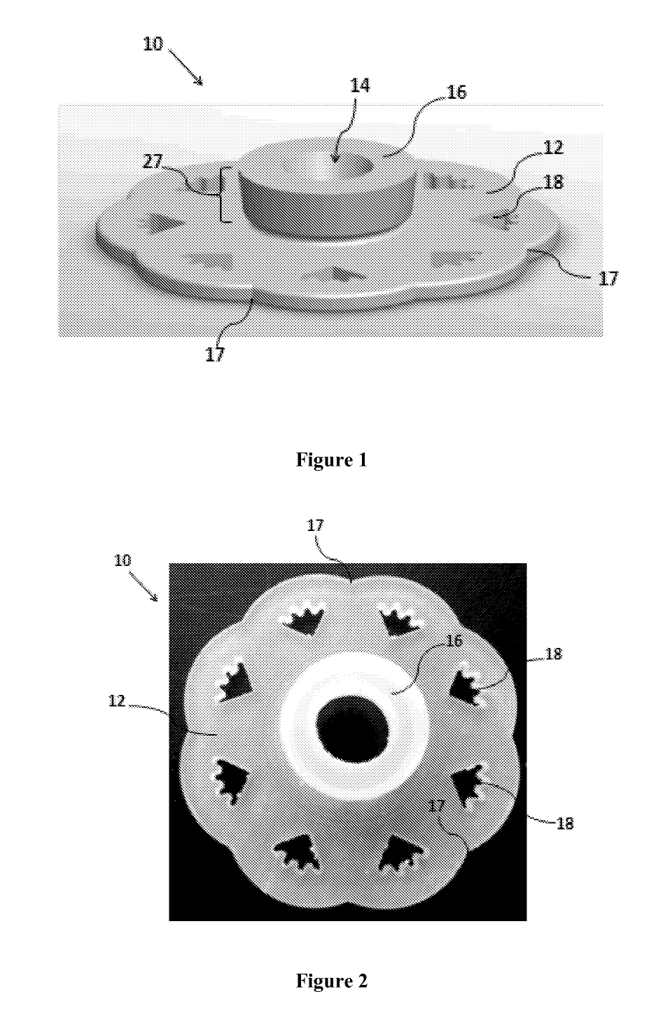

FIG. 1 is a perspective view and FIG. 2 is a top view of one embodiment of a stabilization device 10. As can be seen, the device includes a base 12 and a passage 14 through the base. The device further includes an elevated portion 27 including a supporting wall 16, which surrounds the passage 14. The elevated portion 27 extends from the top surface of the base 21. The height of the elevated portion 27, as measured from the top surface of the base 21 to the top of the elevated portion 20, is optionally from about 5 mm to about 50 mm. For example, the elevated portion is optionally 5 mm, 10 mm, 15 mm, 20 mm, 25 mm, 30 mm, 35 mm, 40 mm, 45 mm, 50 mm, or values between these.

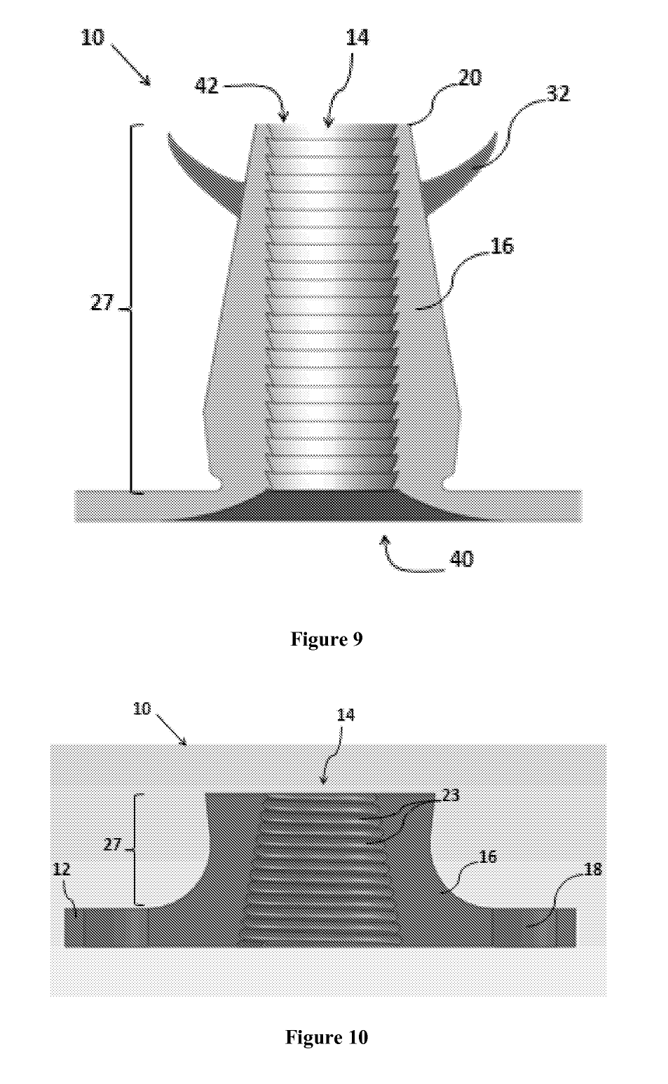

Optionally, as shown in FIG. 9, the supporting member can be of variable thickness as it extends between the base and the top surface of the elevated member. In some embodiments, the supporting member 16 can be tapered, having a larger thickness proximal to the base and a smaller thickness distal to the base. Optionally, the elevated portion 27 further comprises a retaining element 32 positioned in proximity to the top of the elevated portion 20. The retaining element 32 can be for example, a hook, loop, eyelet, or grove which can be used by a physician to secure the top of the elevated portion 20 to the patient, for example, via a suture.

Referring now to FIG. 4, the base 12 and the elevated portion 27 define the passage 14. The passage 14 has an opening at the distal edge of the elevated portion 40 and an opening at the proximal edge of the base portion 42. The largest cross-sectional dimension of the opening at the top of the elevated portion 44 is from about 90% to 99% of the outer diameter of the tube. For example, the percentage is optionally 90%, 91%, 92%, 93%, 94%, 95%, 96%, 97%, 98%, 99%, or percentages between these. The largest cross-sectional dimension of the opening at the top of the elevated portion 44 is optionally from about 0% to 10% smaller than the largest cross-sectional dimension of passageway 14 at the passageway's intersection with the plane formed by the top surface of the base. For example the percentage is optionally about 0%, 1%, 2%, 3%, 4%, 5%, 6%, 7%, 8%, 9%, 10% or percentages between these.

During use, a percutaneous tube can pass through the passage 14 and be supported by the wall 16. The device 10 also includes a series of eyelets 18 that can be used for suture location during anchoring to a patient.

As can be seen, the device 10 can be of a single piece construction and can be formed by use of a single mold and of a uniform material. As such, no assembly of pieces is required during use of the device. This can simplify manufacturing of the device, leading to lower costs, as well as simplify utilization of the device. In general, the device 10 can be formed of a moldable biocompatible, sterilizable polymeric material, such as a silicone elastomer, a polyurethane, or another suitable polymer as is generally known in the art. Optionally, the device is formed of a biocompatible material. For example, the device is optionally formed from a biocompatible silicone elastomer. Optionally, the silicone elastomer has a Shore A hardness of from about 30 to about 60, as measured by DIN 53505. For example, the Shore A hardness is optionally about 30, 35, 40, 45, 50, 55, 60, or hardness values in between these values. Optionally, the silicone elastomer has a tensile strength of from about 6 to about 12 N/mm² as measured by DIN 53504 S 1. For example, the tensile strength is optionally 6, 7, 8, 9, 10, 11, 12 N/mm² or values in between these values. Optionally, the silicone elastomer has a tear strength of from about 20 N/mm to about 70 N/mm, as measured by ASTM D624 B. For example, the tear strength is optionally 20, 25, 30, 35, 40, 45, 50, 55, 60, 65, 70, or values between these values. The device can be formed according to standard methodology, for instance according to an injection molding process as is known.

As seen in FIG. 2, the footprint of the stabilization device 10 can be generally circular, though this is not a requirement and no particular footprint shape is essential for the disclosed devices. For instance, the shape of the footprint can be ovoid, triangular, rectangular, crescent, or any other desired shape. In one embodiment, the base can be specifically designed for a particular anchoring location on the body and the footprint of the device can be such that the device fits at that location. As utilized herein the term ‘footprint’ is intended to refer to the overall shape of the device over that surface of the base that will be secured to the patient during use. The dimensions of the footprint can generally vary depending upon the specific application, e.g., the anchoring site, the expected duration of the insertion, patient size, tube size, etc. By way of example, when considering a generally circular adult chest tube stabilization device, the footprint can have a diameter of from about 10 millimeters to about 70 millimeters, from about 20 millimeters to about 60 millimeters, or from about 30 millimeters to about 50 millimeters.

The device can include features to improve fit between the patient and the device. For instance, in the embodiment illustrated in FIGS. 1 and 2 the device includes a series of indentations 17 around the edge of the base 12 that cause the footprint of the device 10 to be slightly noncircular and form a series of ‘petals’ around the outer edge of the base 12. Such indentations 17 can increase the pliability of the base and improve the ability of the device to contour to the surface of the patient's body during anchoring and thereafter. Improved contouring between the device and the patient can provide for tighter securement during suturing and prevent motion of the inserted percutaneous tube. Of course, while the illustrated device is shown with 8 indentations 17 around the edge of the base 12, this is not a requirement. The size and location of any indentations formed around the edge of a base can be varied as desired. In addition, any indentations need not be located equidistant from one another and need not be of equal size to one another.

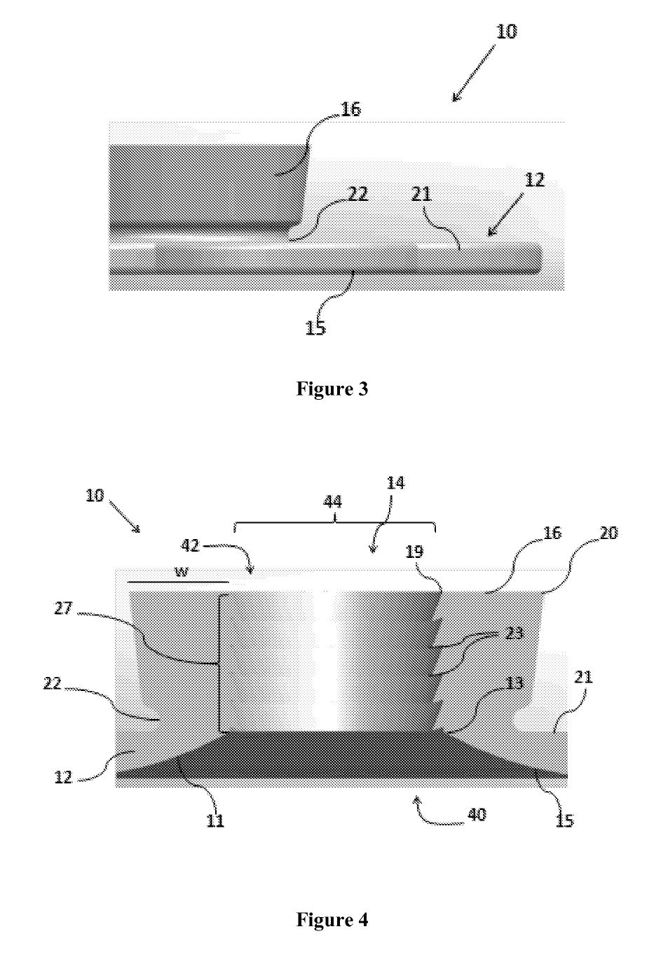

Side views of a device are provided in FIGS. 3 and 4 in which can be seen the supporting wall 16 that extends from the base 12 of the device and surrounds the passage 14. At the outer edge, the lower surface 15 of the base 12 is flat, so as to fit tightly against the patient's skin. As can be seen in the cross sectional view of FIG. 4, near the junction 13 of the lower surface 15 with the passage 14, the lower surface 15 of the base 12 can angle slightly to meet the base of passage 14 at a junction 13. This segment 11 of the base 12 can provide for space between the incision and the lower surface 15 and can also increase flexibility of the device and improve alignment of the device with a percutaneous tube held by the device.

The diameter of the passage 14 can be slightly smaller than the diameter of the percutaneous tube to be held by the device, so as to form an interference fit between the two. The difference in diameter between the outer diameter of the percutaneous tube and the narrowest diameter of the passage 14 can be less than about 1 mm, less than about 0.5 mm, or less than about 0.2 mm, or equivalent tube to diameter ratios, in one embodiment. For example, when a 28 Fr (9.3 mm outer diameter) chest tube is used, the passage 14 can optionally have a narrowest diameter of about 8.9 mm.

In addition, the diameter of the passage 14 can vary somewhat along its length. For instance, the passage 14 can be beveled slightly such that the diameter of the passage 14 increases from the top 19 of the passage 14 to the junction 13 of the passage 14 with the lower surface 15. The slight bevel can generally be less than about 5°, less than about 3°, or less than about 1°, and can ease utilization of the device as well as improve securement and proper exit angle for the percutaneous tube held by the device.

The interior of passage 14 includes one or more textural features. The one or more textural features are optionally configured to engage the tube such that advancement of the tube axially through passage 14 in the direction of the opening at the proximal edge of the base portion 42 produces less resistance than retraction of the tube axially through the passage in the direction of the opening at the distal edge of the elevated portion 40. Optionally, the passage is sized to allow for slideable advancement of the tube axially through the passage in the direction of the opening at the proximal edge of the base portion 42 while the tube is engaged by the textural features.

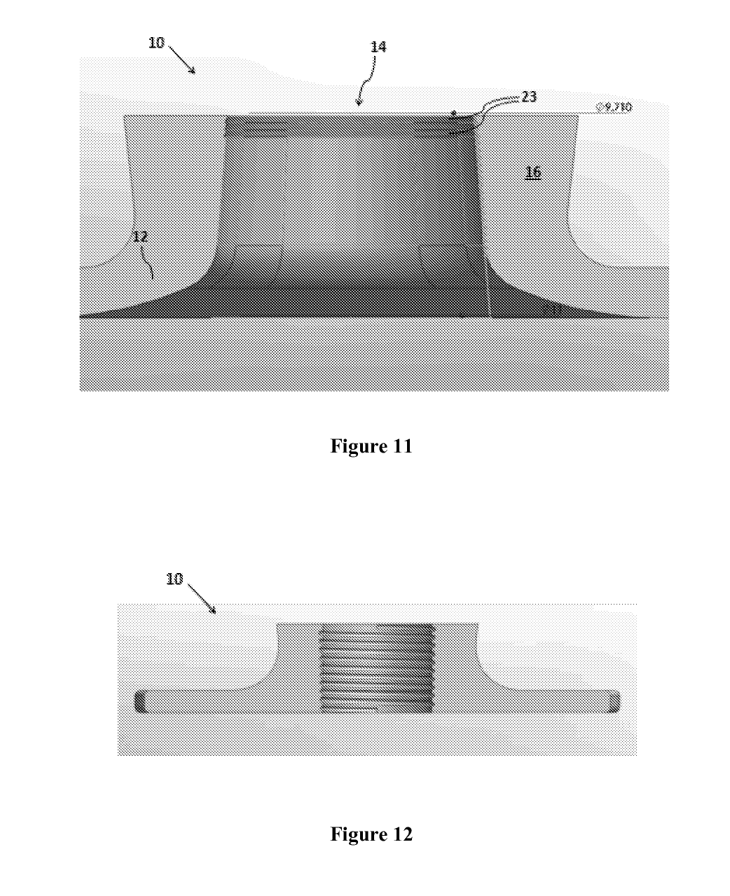

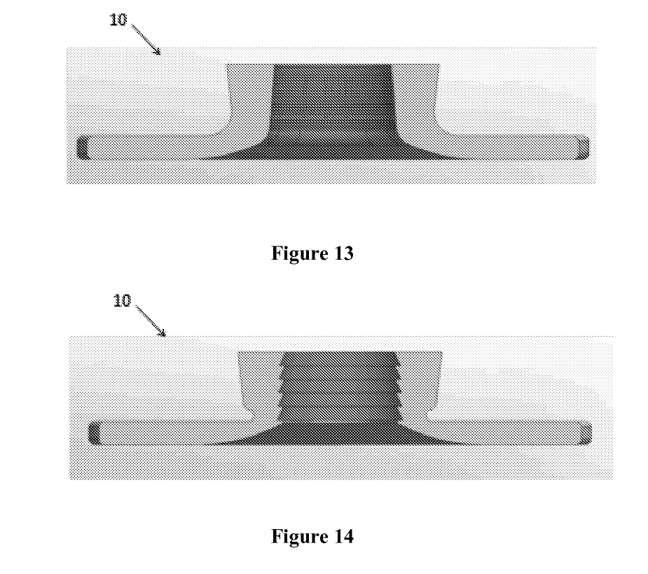

The one or more textural features optionally include one or more protrusions extending from the passage wall into the passage lumen 33, each protrusion including a point in maximal proximity to the central axis of the passage 36. The one or more protrusions are optionally one or more ridges 23. For example, the textural features can comprise a plurality of ridges. The ridges are optionally parallel. The ridges are optionally arranged perpendicular to the central axis of the lumen. The ridges optionally have a pitch relative to the central axis of the lumen, as shown for example in FIG. 12. Optionally, the ridges are continuous around the interior surface of the lumen. Optionally, the ridges are discontinuous around the interior surface of the lumen. As shown in FIG. 11, the ridges 23 can be disposed within a sub-region of the passageway, such as in proximity to the opening in the distal edge of the elevated member.

The relief 37 of the protrusions, measured as the distance from the point in maximal proximity to the central axis 36 to the plane of the interior surface of the passage 34, optionally ranges from about 0.05 mm to about 0.75 mm. For example the distance is optionally about 0.05 mm, 0.10 mm, 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, or values between these. Optionally, the relief 37 of the protrusions, measured as the distance from the point in maximal proximity to the central axis 36 to the plane of the interior surface of the passage 34, is from about 1% to about 10% of the largest cross-sectional dimension of the opening at the top of the elevated portion 40. For example, the percentage is optionally 1%, 2%, 3%, 4%, 5%, 6%, 7%, 8%, 9%, 10% or percentages between these. Optionally, the relief 37 of the protrusions, measured as the distance from the point in maximal proximity to the central axis 36 to the plane of the interior surface of the passage 34, is from about 2.5% to about 8% of the largest cross-sectional dimension of the distal opening of the lumen 40. For example the percentage is optionally about 2.5%, 3.0%, 3.5%, 4.0%, 4.5%, 5.0%, 5.5%, 6.0%, 6.5%, 7.0%, 7.5%, 8.0% or percentages between these values.

Each protrusion optionally includes a base 50 at the passage lumen wall. The base 50 includes a proximal point 52 and a distal point 54. The proximal point 52 is the point at which the base 50 is in maximum proximity to the opening at the bottom of the base portion 42. The distal point 54 is the point at which the base 50 is in maximum proximity to the opening at the top of the elevated portion 40. Optionally, the point in maximal proximity to the central axis 50 is positioned closer to the opening at the bottom of the base portion 42 than the proximal point 52.

The height of the protrusions, measured as the distance from the proximal point 52 to the distal point 54 is optionally from about 0.15 mm to about 1.5 mm. For example, the height is optionally 0.15 mm, 0.20 mm, 0.25 mm, 0.30 mm, 0.35 mm, 0.40 mm, 0.45 mm, 0.55 mm, 0.60 mm, 0.65 mm, 0.70 mm, 0.75 mm, 0.80 mm, 0.85 mm, 0.90 mm, 0.95 mm, 1.0 mm, 1.05 mm, 1.10 mm, 1.15 mm, 1.20 mm, 1.25 mm, 1.30 mm, 1.35 mm, 1.40 mm, 1.45 mm, 1.50 mm, or values between these.

Optionally the textured features, as can be seen in FIG. 4, include a series of ridges 23 on the interior of the passage wall 16. Each ridge can extend in to the passage 14 as shown. Between individual ridges 23 the diameter of the inner surface of the passage 14 can increase, for instance having a diameter that is about equal to or even slightly larger than the diameter of the percutaneous tube to be held by the device. The ridges can further improve the interference fit between the device and a tube held by the device while still maintaining ease of use such that sliding a percutaneous tube into the passage 14 is not difficult.

The various features of a stabilization device at the passage 14 such as one or more of the ridges 23, the segment 11, and the slight bevel of the passage 14 can improve the multiple aspects of the stabilization device 10 during use. For example, a device as illustrated in FIG. 4, which includes all three of these features, can hold a chest tube such that a force of greater than about 5 Newtons (N), for instance greater than about 5.5 N, or about 5.55 N is necessary to dislodge the chest tube from the stabilization device.

The supporting wall 16 defining the passage 14 can extend up from the base 12. For instance, the supporting wall 16 can extend above the upper surface 21 of the base 12 by a distance of from 1 mm to about 50 mm, from about 3 mm to about 40 mm, or from about 5 mm to about 30 mm. The width (W) of the supporting wall 16 should be such that the tube held by the device is properly supported within the passage 14. For instance, the width w can optionally be from about 1 mm to about 10 mm. As can be seen in FIG. 4, the width w of the supporting wall can vary along the height of the wall as well, which can provide increased flexibility to the device so as to allow an amount of supported motion of the percutaneous tube held by the device. For example, the width (W) can be smaller at the top of the elevated portion than at the junction of the elevated portion and the base portion, equal at the top and at the junction, or smaller at the junction than at the top of the elevated portion.

By providing supported motion of a tube, the tube can move a slight amount, for instance an amount of bending can be allowed, without the tube becoming dislodged form the device and also without the device becoming separated from the anchoring site on the patient, either of which can lead to dislodgement of the tube from the patient. The ability of the device to provide an amount of supported motion of a tube held by the device can also increase patient comfort.

Another feature of a stabilization device is a channel 22 that can be at the base of the supporting wall 16. The channel 22 can generally encircle the entire supporting wall, so as to allow an amount of bending motion of the supporting wall 16 without the addition of excessive stress on the tube. This can allow a tube held in the passage 14 to exit from the skin surface at a comfortable angle while avoiding separation of the tube from the stabilization device 10 and also while avoiding excessive stress on sutures holding the stabilization device 10 in place, and thus avoiding separation between the stabilization device 10 and the patient.

The channel 22 at the base of the supporting wall 16 allows for slight bending of the tube for a comfortable exit angle. Stabilizing the percutaneous tube at an exit angle (i.e., with the passage 14 bent away from the normal 90° angle from the base 12 as manufactured) can also increase the pull-out force necessary to dislodge the inserted tube. For instance, when a chest tube exits the body at an angle of 45° and is held by the stabilization device at that angle, the force required to dislodge the chest tube can be greater than about 6 N or greater than about 6.5 N, for instance about 6.7 N.

In clinical use, the common forces experienced by a percutaneous tube such as a chest tube are primarily those of external stress and tension. Percutaneous tubes are usually taped in such a way that they do not significantly twist or stretch, but this can put additional tension on the tube as well as causing kinking of the tube, and this can also decrease the exit angle from the 90° angle that is normal to the skin surface to almost horizontal with the skin surface. The disclosed stabilization device can support the percutaneous tube at an exit angle of the tube from the skin surface that is less than a 90° angle so as to decrease the passive gravity forces that would be exerted on the tube while still maintaining the tube at an exit angle that is comfortable and does not place excessive tension forces on the tube. As discussed previously, the stabilization device can withstand greater than about 6.5 N of force at a 45° angle, and this can more than withstand dislodgement forces due to passive gravitational forces, which have been estimated at 0.17 N.

To anchor the stabilization device to the skin surface at the site of tube insertion, the device can be sutured to the skin directly over the tube insertion site by use of a series of eyelets 18 that are located along the perimeter of the base 12. The eyelets permit passage of a suture through the device and through the skin. Though illustrated with eight eyelets 18, there is no particular number of eyelets that need be formed on a device. For instance, a device may have only two eyelets, three eyelets, etc., or may have more than eight eyelets. In addition, the eyelets may be spaced equidistant from one another around the perimeter of the base 12 as illustrated in the figures, or may be spaced around the perimeter with an unequal spacing, as desired. In those embodiments in which the device includes a large number of eyelets 18, for instance more than about five eyelets, such as the eight eyelets 18 as illustrated in FIG. 2, a surgeon can have increased flexibility to use as many or as few of the eyelets as is necessary to properly secure the device to the skin.

The eyelets 18 can be located at a distance from the passage 14, for instance at a distance of greater than about 1 mm, or greater than about 1.5 mm from the center of the passage 14. Location of the eyelets at a distance from the passage provides for suturing of the device to skin that is at a distance from the incision formed for the insertion. This can further improve the stability of the tube, as the suture sites can be less likely to pull-out as can happen when the sutures are very near the incisions (as is the case for purse string and mattress sutures) as the tissue very near the insertion site can be more easily subjected to degradation due to infection and necrosis. In addition, sutures very near the incision can be subjected to additional tube pressure, and movement of the tube can cause trauma within the body as well as to the surrounding subcutaneous tissue via the sutures, thus causing bruising, hematoma, etc.

Utilization of eyelets for suturing the device to the patient skin surface can also allow for air flow under and around the device, which can improve patient comfort as well as decrease infection risk.

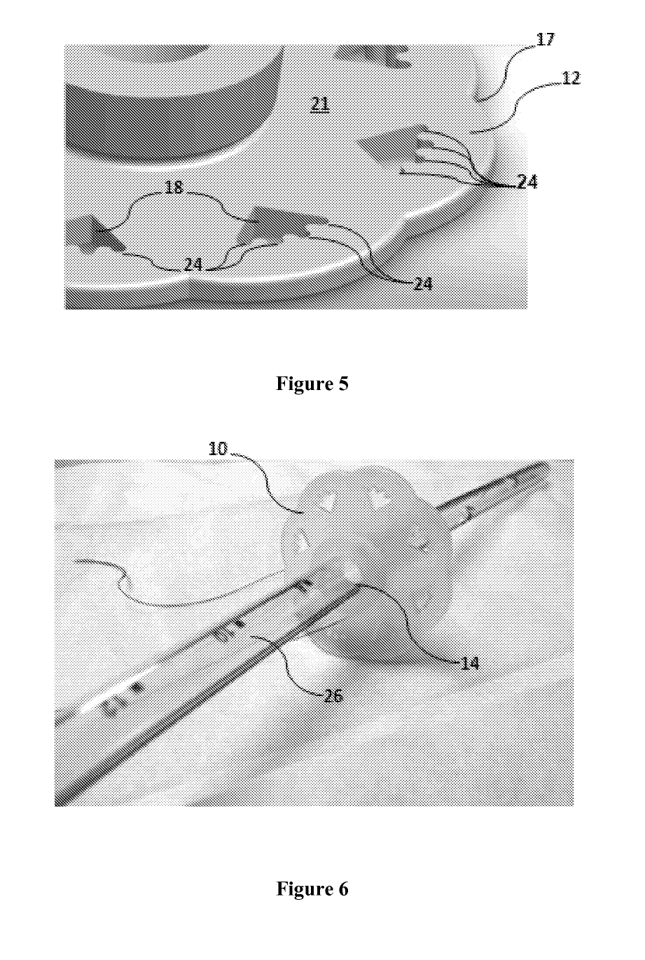

As illustrated in FIG. 5, each eyelet 18 can include a series of insets 24 that provide distinct resting sites for the sutures. During anchoring, the suture can be set within an inset 24. The insets 24 can have rounded edges, which can eliminate sharp corners that can become areas of high stress concentration. In addition, through location of the suture within an inset 24, relative motion between the suture and the base 12 will be decreased and the device 10 will be held more firmly, preventing any rotation that can lead to dislodgement. A suture held within an inset 24 of a device 10 has been shown to withstand an average load of about 12 N. Thus, the suture eyelets 18 including the insets 24 can properly withstand the forces generated during a typical insertion and stabilization procedure, e.g., a thoracostomy procedure.

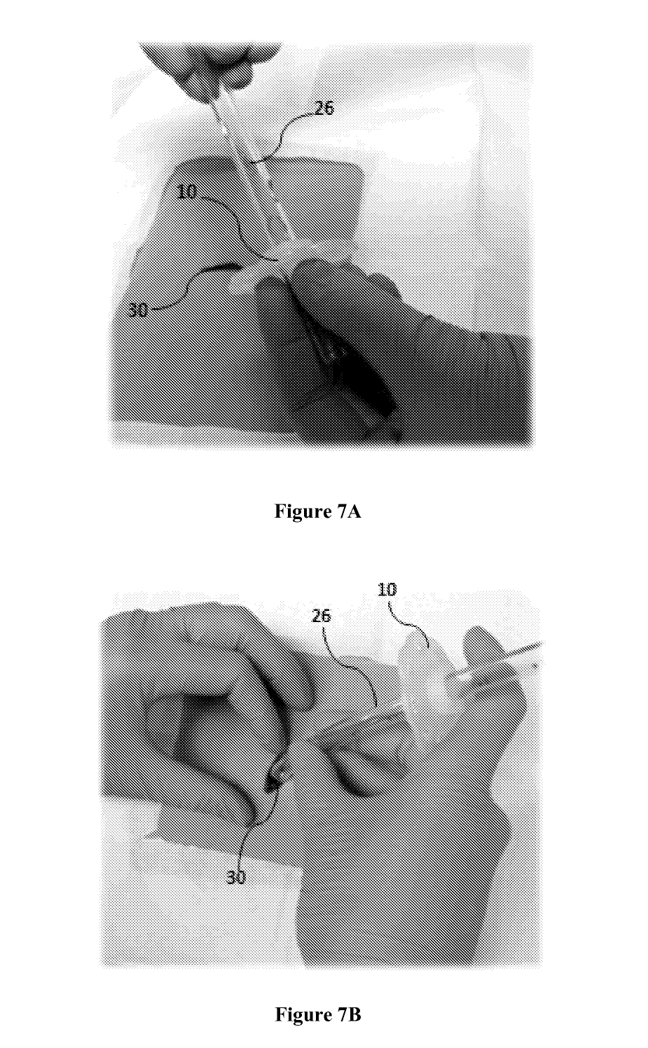

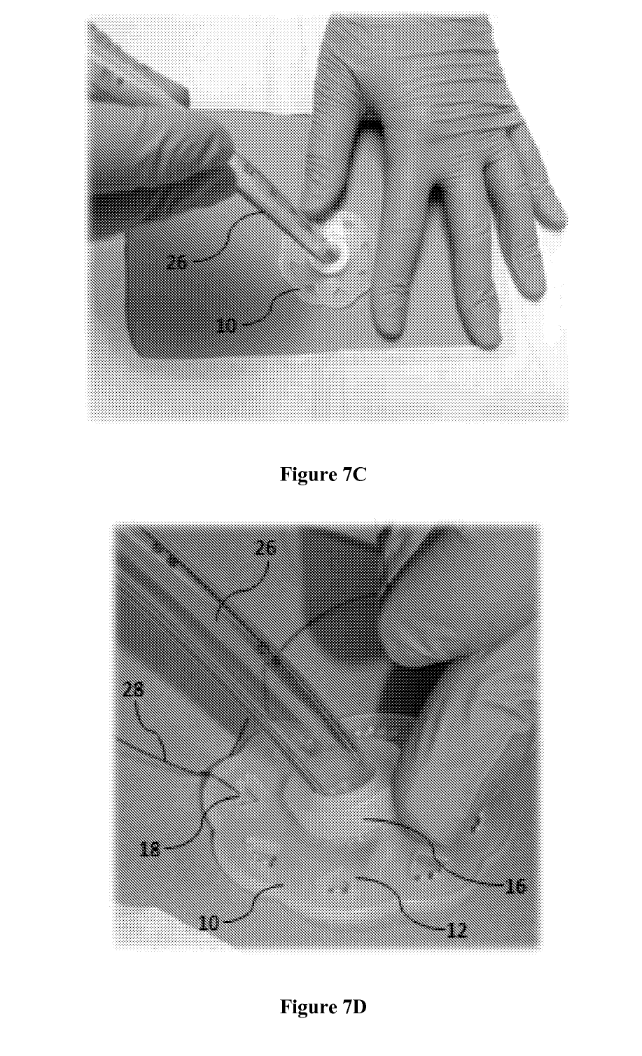

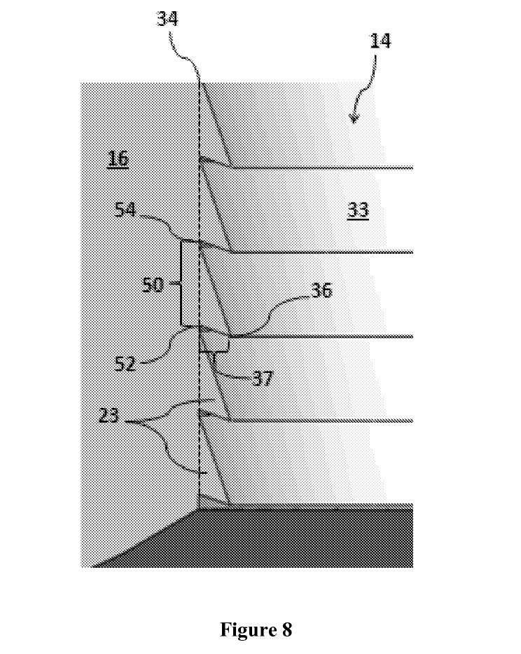

As illustrated in FIG. 6, a device 10 can hold a percutaneous tube 26 within the passage 14 of the device. The device 10 can be slid to the desired location along the tube 26 with an interference fit either prior to or following insertion of the tube. By way of example, FIG. 7 illustrates a thoracostomy procedure in which a chest tube is secured by use of a stabilization device 10. Initially, as shown at FIG. 7A, an insertion site 30 can be formed in the chest wall and the stabilization device 10 can be slid over the chest tube 26 to the approximate site of stabilization. At FIG. 7B the distal end of the chest tube 26 is inserted through the insertion site 30 to the desired depth within the pleural cavity. Following, the device 10 can be moved along the tube 26 as necessary to the skin surface, where it can conform to the surface of the skin, as shown at FIG. 7C. The device 10 can then be sutured to the skin surface (FIG. 7D) by use of suture 28 passed through the eyelet 18 and set into an inset 24 of the eyelet 18 for more secure attachment. As shown in FIG. 7D, the chest tube can have an exit angle from the chest that is less than 90° and the support wall 16 can bend to accommodate this exit angle without pulling the base 12 away from the skin surface. The chest tube 26 can be utilized to drain air, blood, bile, spinal or cranial fluid, pus, or other fluids from the subcutaneous location. Whether the accumulation of fluid is the result of rapid traumatic filling or insidious malignant seepage, placement of the percutaneous tube 26 and stabilization of the percutaneous tube with the stabilization device 10 can allow for continuous, large volume drainage without dislodgment of the tube until the underlying pathology can be more formally addressed.

One or more of the features of the percutaneous tube 10 can be used at the surgeon's discretion in association with other procedural aspects as are generally known. For example, if deemed necessary by the surgeon, an adhesive bandage can be applied to the external portion of the support wall 16 and the percutaneous tube at the exit of the passage 14, to bridge any gap between the device and tube, for instance if the widest portion of the passage 14 has a diameter that is slightly greater than the outside diameter of the percutaneous tube. In one embodiment, an anti-bacterial gel can be applied directly to the bottom surface 15 of the base 12 that is located against the skin surface, so as to further prevent bacterial infection. These are examples of variations in procedure as may be utilized and other variations are well within the knowledge of one of skill in the art.

Disclosed are the components to be used to prepare the disclosed devices as well as the devices themselves to be used within the methods disclosed herein. These and other materials are disclosed herein, and it is understood that when combinations, subsets, interactions, groups, etc. of the components making up a device are disclosed that while specific reference of each various individual and collective combination and permutation of these components may not be explicitly disclosed, each is specifically contemplated and described herein. Thus, if a device formed from A, B, and C is disclosed as well as a components D, E, and F and an example of a combination, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application, including particularly the components of the devices described herein.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present invention.

1. A device for stabilizing a tube in the body of a subject, comprising
(a) a base portion having a proximal patient contacting surface and a top surface;
(b) an elevated portion extending from the base portion; and
(c) a passage defined by the base and elevated portions and having a passage wall, the passage extending from an opening at the distal edge of the elevated portion to an opening at the proximal edge of the base portion, wherein the interior of the passage comprises one or more textural features.

2. The device of claim 1, wherein the one or more textural features are configured to engage the tube such that advancement of the tube axially through the passage in the direction of the opening at the proximal edge of the base portion produces less resistance than refraction of the tube axially through the passage in the direction of the opening at the distal edge of the elevated portion.

3. (canceled)

4. (canceled)

5. The device of claim 1, wherein the device is formed from a biocompatible silicone elastomer, and the silicone elastomer has a Shore A hardness of from about 30 to about 60, as measured by DIN 53505.

6. The device of claim 1, wherein the device is formed from a biocompatible silicone elastomer, and the silicone elastomer has a tensile strength of from about 6 to about 12 N/mm² as measured by DIN 53504 S 1.

7. The device of claim 1, wherein the device is formed from a biocompatible silicone elastomer, and the silicone elastomer has a tear strength of from about 20 N/mm to about 70 N/mm, as measured by ASTM D624 B.

8. The device of claim 1, wherein the passage lumen has a circular transverse cross-section.

9. The device of claim 1, wherein the one or more textural features comprise one or more protrusions extending from the passage wall into the passage lumen, each protrusion including a point in maximal proximity to the central axis of the passage.

10. The device of claim 9, wherein the one or more protrusions comprise one or more ridges.

11. (canceled)

12. (canceled)

13. (canceled)

14. The device of claim 10, wherein the ridges have a pitch relative to the axis of the lumen.

15. The device of claim 10, wherein the ridges are continuous around the interior surface of the lumen.

16. (canceled)

17. The device of claim 9, wherein the relief of the protrusions, measured as the distance from the point in maximal proximity to the central axis to the plane of the interior surface of the passage, ranges from about 0.05 mm to about 0.75 mm

18. The device of claim 9, wherein the relief of the protrusions, measured as the distance from the point in maximal proximity to the central axis to the plane of the interior surface of the passage, is from about 1% to about 10% of the largest cross-sectional dimension of the opening at the top of the elevated portion.

19. The device of claim 9, wherein the relief of the protrusions, measured as the distance from the point in maximal proximity to the central axis to the plane of the interior surface of the passage, is from about 2.5% to about 8% of the largest cross-sectional dimension of the distal opening of the lumen.

20. The device of claim 9, wherein each protrusion comprises a base at the passage lumen wall, the base including a proximal point and a distal point,
wherein the proximal point is the point at which the base is in maximum proximity to the opening at the bottom of the base portion;
wherein the distal point is the point at which the base is in maximum proximity to the opening at the top of the elevated portion; and
wherein the point in maximal proximity to the central axis is positioned closer to the opening at the bottom of the base portion than the proximal point.

21. The device of claim 18, wherein the height of the protrusions, measured as the distance from the proximal point to the distal point is from about 0.15 mm to about 1.5 mm.

22. (canceled)

23. The device of claim 1, wherein the largest cross-sectional dimension of the opening at the top of the elevated portion is from about 90% to about 99% of the outer diameter of the tube.

24. The device of claim 1, wherein the largest cross-sectional dimension of the opening at the distal edge of the elevated portion is from about 0% to about 10% smaller than the largest cross-sectional dimension of the passageway at the passageway's intersection with the plane formed by the top surface of the base.

25. (canceled)

26. The device of claim 1, wherein the central axis of the lumen is angled with respect to the plane of the base portion.

27. The device of claim 1, wherein the base portion further comprises a plurality of anchor points.

28. (canceled)

29. (canceled)

30. (canceled)

31. (canceled)

32. (canceled)

33. (canceled)

34. (canceled)

35. (canceled)

36. The device of claim 1, wherein the passage is sized to allow for slideable advancement of the tube axially through the passage in the direction of the opening at the bottom of the base portion while the tube is engaged by the textural features.

37. (canceled)

38. (canceled)

39. (canceled)

40. (canceled)
